(19) United States
(12) Patent Application Publication
(10) Pub. No.: US 2012/0302931 A1
(43) Pub. Date: Nov. 29, 2012

(54) SPLINT FOR ORTHOPEDICS

(75) Inventor: Ha Ryoung Yu, Seoul (KR)

(73) Assignee: JOIN ENTERPRISE LTD., Yongin-si (KR)

(21) Appl. No.: 13/302,015

(22) Filed: Nov. 22, 2011

(30) Foreign Application Priority Data
Feb. 17, 2011 (KR) 10-2011-0014007
Feb. 17, 2011 (KR) 10-2011-0014011
Sep. 29, 2011 (KR) 10-2011-0098734

(51) Int. Cl. A61F 5/00

(52) U.S. Cl. 602/6

(57) ABSTRACT

A splint for orthopedics with a mid-layer material made from a stretchable woven fabric knitted with a double raschel warp knitting machine and impregnated with a water-curable resin; and top and bottom surface layers consisting of a non-woven fabric stretchable in both horizontal and vertical directions and a barrier layer against the water-curable resin. The stretchable woven fabric is knitted with a double raschel warp knitting machine, which comprises six symmetrically arranged warpers beams, wherein the outer beams hold coarse polyester yarns; the inner beams hold fine polyester yarns, and the central beams hold rubber threads. The barrier layer includes a paper material susceptible to tearing or a synthetic resin film susceptible to stretching while applying the splint. In another embodiment, as the top and bottom surface layers, a paper material susceptible to tearing or a synthetic resin film susceptible to stretching while applying the splint may be used.

### CROSS-REFERENCE TO RELATED APPLICATION

This application claims priorities from Korean Patent Application No. 2011-0014007 filed in the Korean Intellectual Property Office on Feb. 17, 2011, Korean Patent Application No. 2011-0014011 filed in the Korean Intellectual Property Office on Feb. 17, 2011 and Korean Patent Application No. 2011-0098734 filed in the Korean Intellectual Property Office on Sep. 29, 2011, the entire disclosure of each of which is incorporated herein by reference.

### FIELD OF THE INVENTION

The present invention relates to a splint for orthopedics used for the fracture of an injured person and, more particularly, to a splint with good elasticity and resiliency that, when applied to a joint such as an elbow joint or an ankle joint, does not wrinkle on the fold but remains almost in the same shape as the profile of the body part along the contour of the joint.

### BACKGROUND OF THE INVENTION

A splint is a typical orthopedic device that has been applied as a support for the fracture of an injured person. The splint is an orthopedic medical tool used to make swelling and fever on the fracture subside and hold the fracture in position prior to cast application.

To make a splint, top and bottom surface layers are hermetically bonded to the top and bottom sides of a mid-layer material provided in the form of a fabric impregnated with a water-curable resin. When using the splint for a fracture, the splint is removed of the wrapping paper, soaked with water and put on the fracture, so it can harden in the same shape of the fracture.

As the water-curable resin hardens in contact with water, the splint should be stored in a waterproof packing to avoid water penetration.

The quality of the sprint depends on the two factors: the one factor is good elasticity and resiliency that allow the splint to harden in a same shape as the profile of the fracture to be splinted; and the other is no leakage of the water-curable resin from the mid-layer material.

There have been applied many patents and utility model patents in an attempt to developing a splint with good elasticity and resiliency. As the storage period of the splint is 3 years, a variety of techniques have been adopted to prevent a leakage of the water-curable resin from the mid-layer material during the period.

Korean Utility Model Application No. 2002-8609 discloses a splint using tubular stretchable woven fabrics as derived to improve elasticity. Korean Utility Model Application No. 2003-19356 also discloses a splint that includes a mid-layer material using a woven fabric and hydrophilic paddings provided on the top and bottom sides of the mid-layer material to prevent a leakage of the water-curable resin. Korean Patent Application No. 2007-52206 specifies a splint for orthopedics that includes a mid-layer material using single-layered warp knitting woven fabrics made from a polyester DTY fibre with a raschel knitting machine, to enhance elasticity and resiliency.

When applied to a fracture, the above-described conventional splints, with poor elasticity and resiliency, do not harden firmly adherent to the body part but make wrinkles.

Accordingly, the inventor of the present invention has developed a stretchable woven fabric with good elasticity in horizontal and vertical directions as made with a double raschel warp knitting machine for use as a mid-layer material, and a non-woven fabric stretchable in both horizontal and vertical directions as top and bottom surface layers (upper and lower sheets), and provided a barrier layer against the water-curable resin of the mid-layer material between the mid-layer material and the stretchable non-woven fabric, thereby completing a splint having good elasticity and resiliency and highly resisting a leakage of the water-curable resin. Further, the inventor of the present invention has derived a splint that includes a mid-layer material using the stretchable woven fabric, and top and bottom surface layers using a paper material susceptible to tearing or a synthetic resin film susceptible to stretching during an operation of applying the splint to a fracture, thereby maintaining good elasticity and resiliency and securing good resistance to a leakage of the water-curable resin.

### OBJECTS OF THE INVENTION

It is an object of the present invention to provide a splint for orthopedics with good elasticity and resiliency.

It is another object of the present invention to provide a novel stretchable woven fabric as a mid-layer material (3) for preparation of the splint for orthopedics, the stretchable woven fabric having good elasticity in horizontal and vertical directions and being knitted with a double raschel warp knitting machine.

It is still another object of the present invention to provide a novel non-woven fabric stretchable in both horizontal and vertical directions for use as top and bottom surface layers (upper and lower sheets) (1, 2).

It is still further another object of the present invention to provide a splint having a barrier layer against a water-curable resin applied/impregnated in a mid-layer material between the mid-layer material and a stretchable non-woven fabric to maintain good elasticity and resiliency and provide a good resistance to a leakage of the water-curable resin.

It is still further another object of the present invention to provide a splint using, as top and bottom surface layers (upper and lower sheets) (1, 2), a paper material susceptible to tearing or a synthetic resin film susceptible to stretching during an operation of applying the splint to a fracture, to maintain good elasticity and resiliency and provide a good resistance to a leakage of the water-curable resin.

It is still further another object of the present invention to provide a method for manufacturing a splint having good elasticity and resiliency, and a high resistance to a leakage of the water-curable resin.

The above and other objects of the present invention are to be achieved with the present invention that will be described as follows.

### SUMMARY OF THE INVENTION

In accordance with an embodiment of the present invention, there is provided a splint for orthopedics comprising: a mid-layer material (3) made from a stretchable woven fabric knitted with a double raschel warp knitting machine and impregnated with a water-curable resin; and top and bottom surface layers (1, 2) formed on the top and bottom surfaces of the mid-layer material (3) and comprising a non-woven fabric stretchable in both horizontal and vertical directions and a barrier layer against the water-curable resin, the barrier layer against the water-curable resin being formed between the mid-layer material (3) and the stretchable non-woven fabric.

The stretchable woven fabric is knitted with a double raschel warp knitting machine, which comprises symmetrically arranged six warpers beams, in which the outer warpers beams, first and sixth beams hold coarse denier polyester yarns; the inner warpers beams, second and fifth beams hold fine denier polyester yarns, and the central beams, third and fourth beams hold rubber threads (or called “covering yarns”); and the stretchable woven fabric has a thickness in the range of 2 to 8 mm, a horizontal elongation of 40 to 80%, and a vertical elongation of 30 to 60%. Alternatively, fine denier yarns are used for the first and sixth beams, and coarse denier yarns are for the second and fifth beams. Otherwise, in consideration of the required strength of the woven fabrics, the first, sixth, second and fifth beams are all used to hold either coarse denier yarns or fine denier yarns.

The rubber thread may be natural rubber thread, synthetic rubber thread, spandex, or the like. For example, a 210 denier nylon/covering-blended spandex yarn (50% nylon, 50% covering) is preferred.

Glass fiber, acryl fiber, or carbon fiber may be used instead of the coarse/fine denier polyester yarns. Preferably, the coarse denier yarns are about 500 to 1,000 denier and the fine denier yarns are about 100 to 500 denier.

The stretchable non-woven fabric may consist of, principally, polypropylene or polyester, and optionally, nylon or cotton, which are made into a non-woven fabric by needle punching, spunbond, spunlace, or other techniques and then adhesively bound with covering yarns such as natural thread, synthetic thread, spandex, etc. In making the non-woven fabric, the covering yarn may not be adhered to the non-woven fabric but inserted into the middle of a non-woven fabric. As for the non-woven fabric to which a covering yarn is adhered, the side of the non-woven fabric in adhesive contact with the covering yarn is necessarily to adhere to the mid-layer material (3). Preferably, the non-woven fabric of the present invention has an elastic elongation in either horizontal or vertical direction in the range of 30 to 80%.

The barrier layer against the water-curable resin according to the present invention may include a paper material susceptible to tearing or a synthetic resin film susceptible to stretching during an operation of applying the splint.

In an example of the present invention, the synthetic resin film may be polyethylene film, polypropylene film polyethylene terephthalate film, thermoplastic polyurethane film, or rayon film, and 20 μm or less in thickness.

The splint for orthopedics of the present invention is prepared by stretching (elongating) the stretchable non-woven fabrics in a horizontal or vertical direction, or both in the top and bottom, inserting the mid-layer material (3) between the stretched stretchable non-woven fabrics and binding them by bonding.

In accordance with another embodiment of the present invention, there is provided a splint for orthopedics comprising: a mid-layer material (3) made from a stretchable woven fabric knitted by a double raschel warp knitting machine and impregnated with a water-curable resin; and top and bottom surface layers (1, 2) made from a paper material susceptible to tearing or a synthetic resin film susceptible to stretching during an operation of applying the splint and being adhesively bound to the top and bottom surface of the mid-layer material.

Hereinafter, the present invention will be described in further detail with reference to the accompanying drawings.

The present invention is to provide a novel stretchable woven fabric for use as a mid-layer material (3) in the fabrication of a splint for orthopedics, which has good elasticity in horizontal and vertical directions and is knitted with a double raschel warp knitting machine; a novel non-woven fabric for use as top and bottom surface layers (upper and lower sheets) (1, 2), which is stretchable in both horizontal and vertical directions; and a barrier layer against a water-curable resin applied/impregnated in the mid-layer material between the mid-layer material and the stretchable non-woven fabric, thereby providing a splint for orthopedics with good elasticity and resiliency and a high resistance to a leakage of the water-curable resin.

### BRIEF DESCRIPTION OF THE DRAWINGS

FIG. 1 is a schematic diagram showing an embodiment of a splint applied to a foot and let to harden.

FIG. 2 is a partial exploded perspective view showing the inner layer of the splint according to the present invention.

FIG. 3 is a schematic diagram showing six warpers beams symmetrically arranged with respect to the center of a double raschel warp knitting machine.

FIG. 4(A) is a schematic cross-sectional view showing that an unstretched mid-layer material (3) is joined to stretched stretchable non-woven fabrics by bonding.

FIG. 4(B) is a schematic cross-sectional view showing that the stretchable non-woven fabrics are removed of the stretching force and shrunken to form wrinkles after bonding.

FIG. 5 is a schematic cross-sectional view showing top and bottom surface layers (1, 2) consisting of a paper material or a synthetic resin film bound, by bonding, on both surfaces of the unstretched mid-layer material (3) made from a stretchable woven fabric.

### DETAILED DESCRIPTION OF THE INVENTION

The present invention relates to a splint for orthopedics used for the fracture of an injured person and, more particularly, to a splint with good elasticity and resiliency that, when applied to a joint such as an elbow joint or an ankle joint, does not wrinkle on the fold but remains almost in the same shape as the profile of the body part along the contour of the joint.

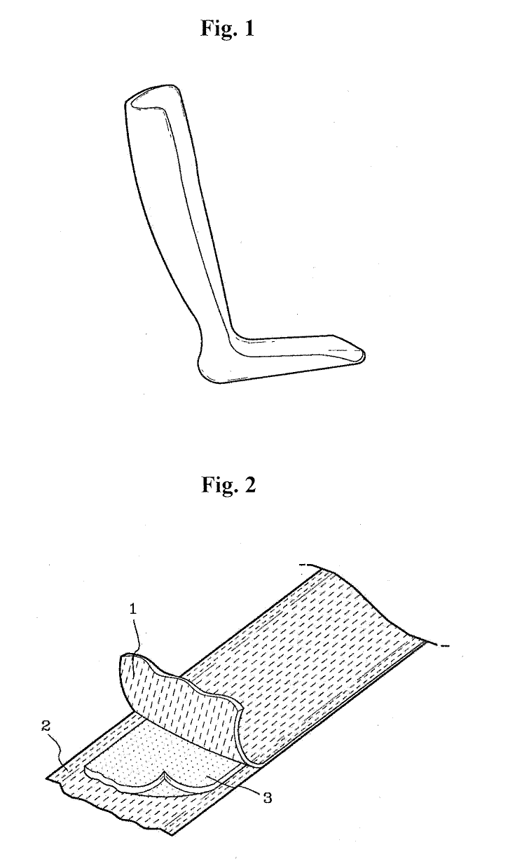

FIG. 1 is a schematic diagram showing an embodiment of a splint applied to a foot and let to harden. FIG. 2 is a partial exploded perspective view showing the inner layer of the splint according to the present invention.

As shown in FIG. 2, the splint includes top and bottom surface layers (1, 2), and a mid-layer material (3) impregnated with a water-curable resin, the mid-layer material (3) being sandwiched between the top and bottom surface layers (1, 2). The top and bottom surface layers (1, 2) are made from a same material, which is a soft material such as a non-woven fabric because the top and bottom surface layers are to be in direct contact with a fractured portion, that is, the injured skin. The mid-layer material (3) impregnated with a water-curable resin is required to have good elasticity and a defined thickness, for it is to be hardened after an operation of applying the splint to the fracture. Generally, the mid-layer material (3) is made from non-woven fabric, multi-layered polyester fabric, glass fiber, or the like that is about 5 to 8 mm thick.

The splint of the present invention includes novel materials for the top and bottom surface layers (1, 2) as well as for the mid-layer material (3).

The splint for orthopedics of the present invention comprises a mid-layer material (3) made from a stretchable woven fabric knitted with a double raschel warp knitting machine and impregnated with a water-curable resin; and top and bottom surface layers (1, 2) formed on the top and bottom surfaces of the mid-layer material (3) and comprising a non-woven fabric stretchable in both horizontal and vertical directions and a barrier layer against the water-curable resin, the barrier layer against the water-curable resin being formed between the mid-layer material (3) and the stretchable non-woven fabric.

The mid-layer material (3) will be first described as follows.

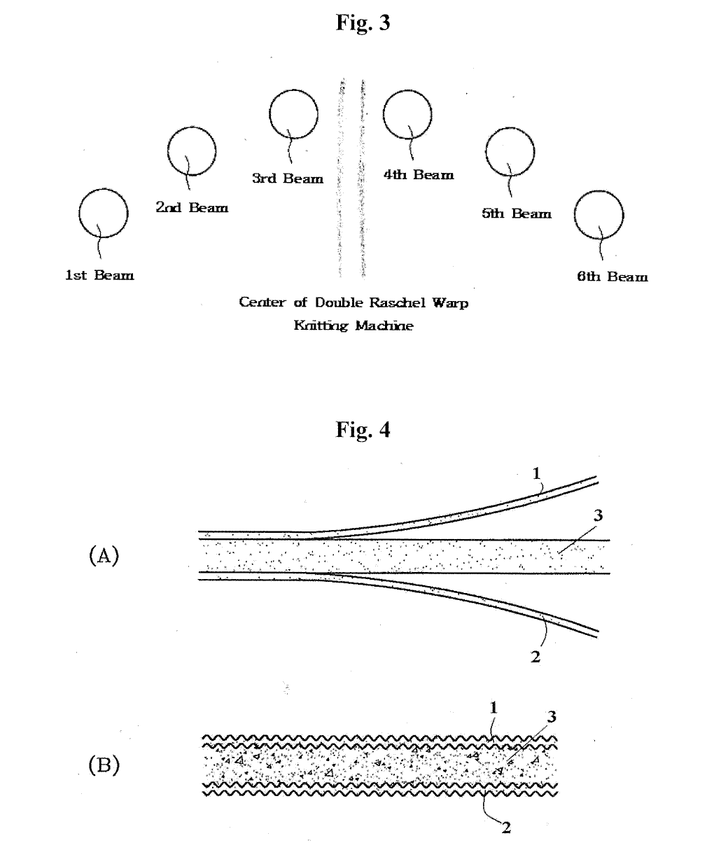

In the present invention, a stretchable woven fabric is knitted with a double raschel warp knitting machine for use as the mid-layer material (3). The stretchable woven fabric is knitted with a double raschel warp knitting machine that consists of six warpers beams symmetrically arranged. FIG. 3 is a schematic diagram showing the positions of six warpers beams symmetrically arranged with respect to the center of a double raschel warp knitting machine.

Referring to FIG. 3, the outer warpers beams, first and sixth beams hold coarse denier polyester yarns; the inner warpers beams, second and fifth beams hold fine denier polyester yarns; and the central beams, third and fourth beams hold rubber threads (or called “covering yarns”). The raschel warp knitting machine or the double raschel warp knitting machine is well understood by those skilled in the art. The knitting technique with six warpers beams in the present invention is also well realized by those skilled in the art.

The coarse denier yarns for the first and sixth beams are about 500 to 1,000 denier, preferably about 600 denier. The fine denier yarns for the second and fifth beams are about 100 to 500 denier, preferably about 300 denier. The rubber thread is natural rubber thread, synthetic rubber thread, spandex, or the like, with a fineness of 100 to 300 denier. For example, a 210 denier nylon/covering-blended spandex yarn (50% nylon, 50% covering) is preferred. Alternatively, fine denier yarns are used for the first and sixth beams, and coarse denier yarns are for the second and fifth beams. Otherwise, in consideration of the required strength of the fabrics, the first, sixth, second and fifth beams are all used for either coarse denier yarns or fine denier yarns.

The stretchable woven fabric knitted under the above-defined conditions has a thickness of 2 to 8 mm, and a good elasticity in both horizontal and vertical directions, with a horizontal elastic elongation of 40 to 80% and a vertical elastic elongation of 30 to 60%. Hence, during an operation of applying the splint to a fracture, the mid-layer material (3) does not make wrinkles along the target injured part but hardens almost in the same shape as the profile of the body part along the contour of the body.

The mid-layer material (3) is impregnated with a water-curable resin. The examples of the water-curable resin include a polyurethane resin, or a reinforced polyurethane resin. The method for impregnating the mid-layer material with the water-curable resin include, for example, soaking the mid-layer material in a liquid water-curable resin, or repeatedly coating the surface of the mid-layer material with a liquid water-curable resin. These methods are well performed by those skilled in the art.

A description will now be given as to the top and bottom surface layers (1, 2), which are formed on the top and bottom surfaces of the mid-layer material (3) and constituted with a non-woven fabric stretchable in both horizontal and vertical directions and a barrier layer against the water-curable resin.

The non-woven fabric derived in the present invention is a non-woven fabric stretchable in both horizontal and vertical directions, preferably with an elastic elongation in the range of 30 to 80% when stretched in a horizontal or vertical direction.

The stretchable non-woven fabric consists of, principally, polypropylene or polyester, and optionally, nylon or cotton, which are made into a non-woven fabric by needle punching, spunbond, spunlace, or other techniques and then bound with covering yarns such as natural thread, synthetic thread, spandex, etc. The technique for preparing the non-woven fabric can be well performed by those skilled in the art. But, when the non-woven fabric is adhesively bound to either one side of the covering yarn, it is required to form the covering yarn on the side of the mid-layer material (3). This is taking into consideration the contact with the skin while applying the splint to the fracture. The covering yarn may not be adhered to the non-woven fabric but inserted into the middle of the non-woven fabric. In this case, either side of the stretchable non-woven fabric can be formed on the side of the mid-layer material (3) to avoid the problem that the stretchable non-woven fabric adhered to only either one side of the covering yarn is ready to curl towards the covering yarn in the course of the fabrication of the splint for orthopedics.

The barrier layer against the water-curable resin according to the present invention is formed between the mid-layer material (3) and the stretchable non-woven fabric to prevent a leakage of the water-curable resin applied/impregnated in the mid-layer material (3) out of the splint for orthopedics and to prevent a water penetration into the mid-layer material (3), thereby avoiding a hardening of the mid-layer material prior to an operation of applying the splint to a fracture. Preferably, the barrier layer is susceptible to breaking during the operation, and thereby does not affect the elasticity and resiliency of the splint for orthopedics.

In an embodiment of the present invention, the barrier layer against the water-curable resin may be formed by bonding. The method of bonding may be any bonding technique that enables the barrier layer to bind the mid-layer material (3) and the stretchable non-woven fabric together, to prevent a leakage of the water-curable resin and a water penetration, and to be susceptible to breaking during an operation of applying the splint.

In an embodiment of the present invention, the barrier layer against the water-curable resin may include a paper material susceptible to tearing, or a synthetic resin film susceptible to stretching during the operation.

The paper material may be any paper that is capable of preventing a leakage of the water-curable resin applied/impregnated in the mid layer material (3) and water penetration and susceptible to tearing during the operation. Used only for preventing a leakage of the water-curable resin prior to the operation, the paper material evenripped off during the operation hardly affects the effect and function of the splint.

Likewise, the synthetic resin film may be any synthetic resin film that is capable of preventing water penetration and a leakage of the water-curable resin applied/impregnated in the mid-layer material (3) and susceptible to tearing or stretching during the operation. In an embodiment of the present invention, the synthetic resin film may be polyethylene film, polypropylene film, polyethylene terephthalate film, thermoplastic polyurethane film, or rayon film. The synthetic resin film, which is required to have good elasticity, is 20 μm or less, preferably 10 μm or less in thickness. For example, the thickness of the synthetic resin film is in the range of 1 to 20 μm, or 5 to 10 μm. Unlike paper materials, the synthetic resin film is not easy to tear but stretchable, and thus required to be equivalent or superior in elasticity to the stretchable woven fabric that is used for the mid-layer material. The synthetic resin film, which is susceptible to stretching instead of tearing, has nothing to do with the effect and function of the splint product. The rayon film is intermediate in properties between the paper material and the synthetic resin film and partly breakable and partly stretchable, so it scarcely affects the effect and function of the splint.

During an operation, the splint is stretchable in any desired direction with good elasticity and also curable in the same shape as the profile of the body with good resiliency. To impart good elasticity and resiliency in the splint, the present invention proposes a novel method for preparing a splint.

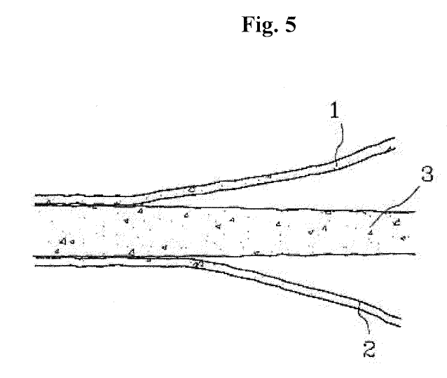

The splint for orthopedics of the present invention is prepared by stretching (elongating) the stretchable non-woven fabrics in a horizontal or vertical direction, or both in the top and bottom, inserting the mid-layer material (3) between the stretched non-woven fabrics and binding them by bonding. FIG. 4(A) is a schematic cross-sectional view showing that an unstretched mid-layer material (3) is joined to stretched stretchable non-woven fabrics by bonding. FIG. 4(B) is a schematic cross-sectional view showing that the stretchable non-woven fabrics are removed of the stretching force and shrunken to form wrinkles after bonding.

Of course, such a process is carried out continuously in automated equipment, to stretch (elongate) the stretchable non-woven fabrics in a horizontal or vertical direction, or both in the top and bottom, insert the mid-layer material (3) between the stretched non-woven fabrics and join them by bonding. Upon removing the stretchable non-woven fabric of the stretching force, as shown in FIG. 4(B), the stretchable non-woven fabric and the mid-layer material shrink to form wrinkles. The formation of wrinkles enhances, during the operation, elasticity better enough to make the splint stretchable or elongated in a desired direction, and improves resiliency to make the splint harden in the same shape as the profile of the body without wrinkles.

The splint for orthopedics may also be prepared by stretching (elongating) the mid-layer material (3) in a horizontal or vertical direction, or both and then binding the stretchable non-woven fabric on top and bottom sides of the mid-layer material (3) by bonding. Alternatively, the splint for orthopedics may be prepared by stretching (elongating) both the mid-layer material (3) and the stretchable non-woven fabric in a horizontal or vertical direction, or both, inserting the stretched mid-layer material (3) between the stretched stretchable non-woven fabric, and then binding the mid-layer material and the non-woven fabric together by bonding.

Binding the stretchable non-woven fabric and the mid-layer material (3) together by bonding is to form a barrier layer against the water-curable resin between the stretchable non-woven fabric and the mid-layer material (3) and thereby to prevent a leakage of the water-curable resin from the stretchable non-woven fabric and a water penetration into the mid-layer material (3), avoiding an undesired hardening of the water-curable resin before the operation.

In an embodiment of the present invention, when the paper material or the synthetic resin film is used as the barrier layer against the water-curable resin, the preparation method for the splint for orthopedics may include, stretching (or not stretching) the mid-layer material (3), and then adhesively binding the stretched or unstretched non-woven fabric, the inner side of which is adhered to the paper material or the synthetic resin film, on the top and bottom sides of the mid-layer material (3). The splint for orthopedics according to another embodiment of the present invention includes a mid-layer material (3) made from a stretchable woven fabric knitted by a double raschel warp knitting machine and impregnated with a water-curable resin; and top and bottom surface layers (1, 2) made from a paper material susceptible to tearing or a synthetic resin film susceptible to stretching during an operation and being adhesively bound to the top and bottom surface of the mid-layer material.

The paper material may be any paper that is capable of preventing a leakage of the water-curable resin applied/impregnated in the mid-layer material (3) and water penetration and susceptible to tearing during the operation. Used only for preventing a leakage of the water-curable resin prior to the operation, the paper material even if ripped off during the operation hardly affects the effect and function of the splint product.

Likewise, the synthetic resin film may be any synthetic resin film that is capable of preventing water penetration and a leakage of the water-curable resin applied/impregnated in the mid-layer material (3) and susceptible to tearing or stretching during the operation. In an embodiment of the present invention, the synthetic resin film may be polyethylene film, polypropylene film, polyethylene terephthalate film, thermoplastic polyurethane film, or rayon film. The synthetic resin film, which is required to have good elasticity, is 20 μm or less, preferably 10 μm or less in thickness. For example, the thickness of the synthetic resin film is in the range of 1 to 20 μm, or 5 to 10 μm. Unlike paper materials, the synthetic resin film is not ready to tear but stretchable, and thus required to be equivalent or superior in elasticity to the stretchable woven fabric that is a material of the mid-layer material. The synthetic resin film, which is susceptible to stretching instead of tearing, has nothing to do with the effect and function of the splint product. The rayon film, which is intermediate in properties between the paper material and the synthetic resin film, is susceptible to party tearing and partly stretching, so it had nothing to do with the effect and function of the splint product.

The splint for orthopedics according to another embodiment of the present invention is prepared by adhesively binding the paper material or the synthetic resin film used as the top and bottom surface layers (1, 2) to both surface sides of the stretchable woven fabric used as the mid-layer material (3) by bonding.

FIG. 5 is a schematic cross-sectional view showing that top and bottom surface layers (1, 2) consisting of a paper material or a synthetic resin film are adhesively bound on both surface sides of the unstretched mid-layer material (3) made from a stretchable woven fabric by bonding.

The splint for orthopedics according to another embodiment of the present invention may be prepared by adhesively binding the paper material or the synthetic resin film to the top and bottom sides of the stretchable woven fabric impregnated with the water-curable resin, with stretching or not stretching the stretchable woven fabric.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A splint for orthopedics comprising:
a mid-layer material made from a stretchable woven fabric knitted with a double raschel warp knitting machine and impregnated with a water-curable resin; and
top and bottom surface layers formed on the top and bottom surfaces of the mid-layer material and comprising a non-woven fabric stretchable in both horizontal and vertical directions and a barrier layer against the water-curable resin, wherein the barrier layer against the water-curable resin is formed between the mid-layer material and the stretchable non-woven fabric.

2. The splint for orthopedics of claim 1, wherein the stretchable woven fabric is knitted with a double raschel warp knitting machine, which comprises symmetrically arranged six warpers beams, in which the outer warpers beams, first and sixth beams hold coarse denier polyester yarns; the inner warpers beams, second and fifth beams hold fine denier polyester yarns, and the central beams, third and fourth beams hold rubber threads;
and the stretchable woven fabric has a thickness in the range of 2 to 8 mm, a horizontal elongation of 40 to 80%, and a vertical elongation of 30 to 60%.

3. The splint for orthopedics of claim 2, wherein the rubber threads are natural rubber thread, synthetic rubber thread, or spandex.

4. The splint for orthopedics of claim 2, wherein the coarse denier yarns of the first and sixth beams are about 500 to 1,000 denier, the fine denier yarns of the second and fifth beams are about 100 to 500 denier, and the rubber threads have a fineness of about 100 to 300 denier.

5. The splint for orthopedics of claim 2, wherein the first and sixth beams hold fine denier yarns of about 100 to 500 denier, the second and fifth beams hold coarse denier yarns of about 500 to 1000 denier, and the rubber threads have a fineness of about 100 to 300 denier.

6. The splint for orthopedics of claim 2, wherein the first, sixth, second and fifth beams hold fine denier yarns of about 100 to 500 denier or coarse denier yarns of about 500 to 1000 denier, and the rubber threads have a fineness of about 100 to 300 denier.

7. The splint for orthopedics of claim 1, wherein the stretchable non-woven fabric comprises polypropylene or polyester or mixtures thereof, which are made into a non-woven fabric by needle punching, spunbond, spunlace, or other techniques and then adhesively bound with natural rubber thread, synthetic rubber thread or spandex covering yarns.

8. The splint for orthopedics of claim 1, wherein the stretchable non-woven fabric is prepared by inserting natural rubber thread, synthetic rubber thread or spandex covering yarns into the middle of a non-woven fabric.

9. The splint for orthopedics of claim 1, wherein the barrier layer against the water-curable resin includes a paper material susceptible to tearing or a synthetic resin film susceptible to stretching during an operation of applying the splint.

10. The splint for orthopedics of claim 9, wherein the synthetic resin film is polyethylene film, polypropylene film, polyethylene terephthalate film, thermoplastic polyurethane film, or rayon film, and has a thickness of 20 μm or less.

11. A method for preparing a splint for orthopedics comprising:
stretching stretchable non-woven fabrics in a horizontal or vertical direction, or both in the top and bottom, wherein the stretchable non-woven fabric comprises polypropylene or polyester or mixtures thereof, which are made into a non-woven fabric by needle punching, spunbond, spunlace, or other techniques and then adhesively bound with natural rubber thread, synthetic rubber thread or spandex covering yarns; and
inserting a mid-layer material made from a stretchable woven fabric knitted with a double raschel warp knitting machine and impregnated with a water-curable resin between the stretched stretchable non-woven fabrics and binding them by bonding.

12. A stretchable woven fabric for use as a mid-layer material of a splint for orthopedics knitted with a double raschel warp knitting machine, which comprises symmetrically arranged six warpers beams, in which the outer warpers beams, first and sixth beams hold coarse denier polyester yarns; the inner warpers beams, second and fifth beams hold fine denier polyester yarns, and the central beams, third and fourth beams hold rubber threads, wherein the stretchable woven fabric has a thickness in the range of 2 to 8 mm, a horizontal elongation of 40 to 80%, and a vertical elongation of 30 to 60%.

13. A splint for orthopedics comprising:
a mid-layer material made from a stretchable woven fabric knitted by a double raschel warp knitting machine and impregnated with a water-curable resin; and
top and bottom surface layers made from a paper material susceptible to tearing or a synthetic resin film susceptible to stretching during an operation of applying the splint and being adhesively bound to the top and bottom surface of the mid-layer material.

14. The splint for orthopedics of claim 13, wherein the stretchable woven fabric is knitted with a double raschel warp knitting machine, which comprises symmetrically arranged six warpers beams, in which the outer warpers beams, first and sixth beams hold coarse denier polyester yarns; the inner warpers beams, second and fifth beams hold fine denier polyester yarns, and the central beams, third and fourth beams hold rubber threads; and the stretchable woven fabric has a thickness in the range of 2 to 8 mm, a horizontal elongation of 40 to 80%, and a vertical elongation of 30 to 60%.

15. The splint for orthopedics of claim 14, wherein the rubber threads are natural rubber thread, synthetic rubber thread, or spandex.

16. The splint for orthopedics of claim 14, wherein the coarse denier yarns of the first and sixth beams are about 500 to 1,000 denier, the fine denier yarns of the second and fifth beams are about 100 to 500 denier, and the rubber threads have a fineness of about 100 to 300 denier.

17. The splint for orthopedics of claim 14, wherein the first and sixth beams hold fine denier yarns of about 100 to 500 denier, the second and fifth beams hold coarse denier yarns of about 500 to 1000 denier, and the rubber threads have a fineness of about 100 to 300 denier.

18. The splint for orthopedics of claim 14, wherein the first, sixth, second and fifth beams hold fine denier yarns of about 100 to 500 denier or coarse denier yarns of about 500 to 1000 denier, and the rubber threads have a fineness of about 100 to 300 denier.

19. The splint for orthopedics of claim 13, wherein the paper material is capable of preventing a leakage of the water-curable resin impregnated in the mid-layer material and water penetration and susceptible to tearing during an operation of applying the splint.

20. The splint for orthopedics of claim 13, wherein the synthetic resin film is polyethylene film, polypropylene film, polyethylene terephthalate film, thermoplastic polyurethane film, or rayon film, and has a thickness of 20 μm or less.
